(19) United States
(12) Patent Application Publication
(10) Pub. No.: US 2022/0047182 A1
(43) Pub. Date: Feb. 17, 2022

(54) INFORMATION PROCESSING DEVICE, INFORMATION PROCESSING METHOD AND RECORDING MEDIUM

(71) Applicant: CASIO COMPUTER CO., LTD., Tokyo (JP)

(72) Inventor: Takehiro AIBARA, Tokyo (JP)

(73) Assignee: CASIO COMPUTER CO., LTD., Tokyo (JP)

(21) Appl. No.: 17/373,474

(22) Filed: Jul. 12, 2021

(30) Foreign Application Priority Data
Aug. 14, 2020 (JP) 2020-136893

(51) Int. Cl. A61B 5/11; A61B 5/00; G16H 40/67; G16H 50/30

(52) U.S. Cl. CPC A61B 5/112; A61B 5/7278; A61B 2562/0219; G16H 50/30; G16H 40/67

(57) ABSTRACT

An information processing device includes a processor that acquires a peak in secondary motion data for each of predetermined periods based on the secondary motion data. The secondary motion data indicates a level of primary motion data obtained from running or walking of a subject. The processor derives an index. of a landing impact of the subject for each of the predetermined periods based on (i) the acquired peak in the secondary motion data and (ii) a landing time of the subject which is obtained from the primary motion data.

### CROSS REFERENCE TO RELATED APPLICATION

This application is based upon and claims the benefit of priority under 35 USC 119 of Japanese Patent Application No. 2020-136893 filed on Aug. 14, 2020, the entire disclosure of which, including the description, claims, drawings and abstract, is incorporated herein by reference in its entirety.

### BACKGROUND

### 1. Technical Field

The present invention relates to an information processing device, an information processing method, and a recording medium.

### 2. Description of Related Art

For example, JP 2001-29329A filed in Japan discloses a floor reaction. force measurement device. In the device, a distribution measurement sheet is put on an upper side of a force plate. The distribution measurement sheet measures distribution of a reaction force on the force plate.

### SUMMARY OF INVENTION

To achieve at least one of the above-mentioned objects, according to an aspect of the present invention, information processing device includes at least one processor that:

acquires a peak in secondary motion data for each of predetermined periods based on the secondary motion data indicating a level of primary motion data obtained from running or walking of a subject; and

derives an index of a landing impact of the subject for each of the predetermined periods based on:
the acquired peak in the secondary motion data; and
a landing time of the subject which is obtained from the primary motion data.

### BRIEF DESCRIPTION OF DRAWINGS

The advantages and features provided by one or more embodiments of the invention will become more fully understood from the detailed description given hereinbelow and appended drawings which are given by way of illustration only, and thus are not intended as a definition of the limits of the present invention.

FIG. 1 is a block diagram showing a running analysis system of an embodiment.

FIG. 2 shows a state in which the measurement device is worn by a user.

FIG. 3 is a block diagram showing functional configuration of the measurement device.

FIG. 4 is a block diagram showing functional configuration of a running analysis device.

FIG. 5 is a graph showing result of measurement of a reaction force when a foot of a running subject lands on a force plate.

FIG. 6 shows three force plate indexes VIP, VALR, and VILR of a landing impact.

FIG. 7 is a graph that shows a waveform showing a reaction force in an up-down direction together with a norm waveform showing norms of reaction forces in three directions (right-left direction, back-forth direction, and. up-down direction).

FIG. 8 is a flowchart showing control procedure of running index deriving processing.

FIG. 9 shows a waveform of acceleration data on which coordinate transformation processing into a world coordinate system is performed.

FIG. 10 is a graph that shows a waveform of acceleration norm data together with a force plate waveform.

FIG. 11 shows a method. of deriving a running index.

FIG. 12 is a flowchart showing control procedure of estimation processing of a waveform indicating a ground reaction force.

FIG. 13 shows a method of revising a waveform of the acceleration norm data.

FIG. 14 shows the waveform. of the acceleration norm data after revision.

FIG. 15 shows a method of estimating a landing impact component and a propulsion component of the ground reaction force.

FIG. 16 is a graph showing an approximate waveform of the landing impact component and an approximate waveform of the propulsion component.

FIG. 17 is a graph showing an estimated force plate waveform and a force plate waveform.

FIG. 18 is a graph snowing an approximate waveform of the landing impact component and an approximate waveform of the propulsion component.

FIG. 19 is a graph showing an estimated force plate waveform and the force plate waveform.

### DETAILED DESCRIPTION OF EMBODIMENTS

Hereinafter, one or more embodiments of the present invention will be described with reference to the drawings. However, the scope of the invention is not limited. to the disclosed embodiments.

### Running Analysis System

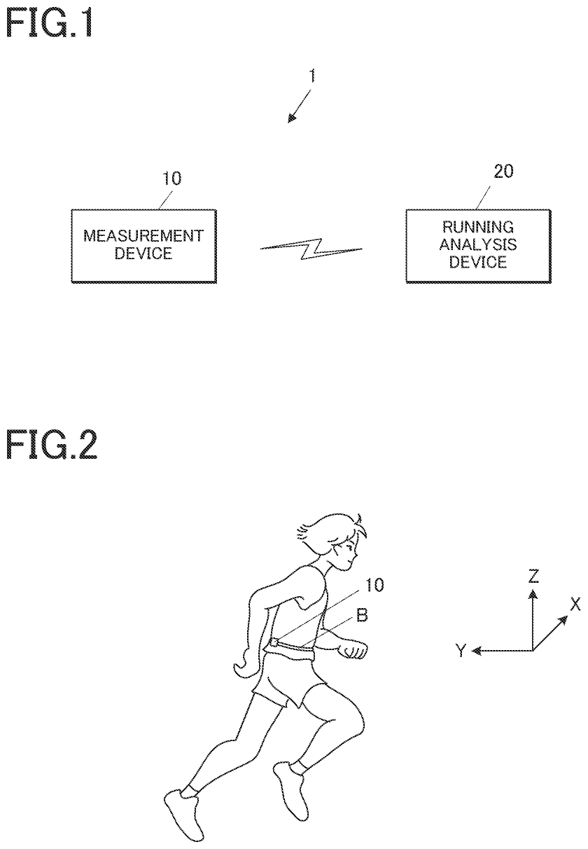

Configuration of an embodiment will be described with reference to FIGS. 1 to 2. First, a running analysis system 1 of the embodiment will be described with reference to FIG. 1.

FIG. 1 is a block diagram showing the running analysis system 1 of the embodiment.

As shown in FIG. 1, the running' analysis system 1 includes a measurement device (information processing device) 10 and a running analysis device (external device) 20.

The measurement device 10 is worn by a target person (user, runner, etc.) in running training or a race. The measurement device 10 collects motion data in the training or the race, such as acceleration data and angular velocity data. The measurement device 10 records running index data derived from the motion data. The measurement device 10 includes, for example, a belt B attached to the measurement device 10 as shown in. FIG. 2. The belt B fixes the measurement device 10 at a position of a user's waist (sacrum).

The measurement device 10 may include a clip instead of the belt B. The clip sandwiches a user's running wear so that the measurement device 10 is fixed at a position of a user's waist.

The running analysis device 20 displays the running index data of the user which is acquired from the measurement device 10. The running analysis device 20 is, for example, a smart watch, a smartphone, or a tablet PC. In the following description, the running analysis device 20 is a smart watch.

### Measurement Device

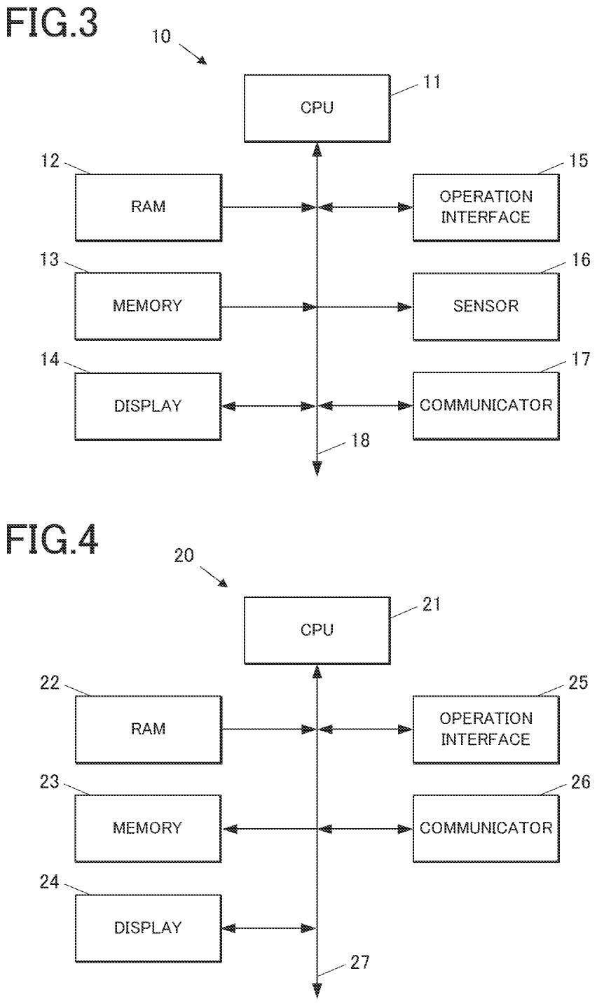

Next, functional configuration of the measurement device 10 will be described with reference to FIG. 3. FIG. 3 is a block diagram showing the functional configuration of the measurement device 10.

As shown in FIG. 3, the measurement device 10 includes a CPU (central processing unit) 11, RAM (random access memory) 12, memory 13, a display 14, an operation interface 15, a sensor 16, and a communicator 17. Components of the measurement device 10 are connected via. a bus 18.

The CPU (processor) 11 controls the components of the measurement device 10. The CPU 11 reads a designated program among system programs and application programs stored in the memory 13 and develops them in the RAM 12. The CPU cooperates with the programs to execute processing of various kinds.

The RAM 12 is volatile memory and provides a work area where various data and programs are temporarily stored.

The memory 13 is constituted by flash memory, EEPROM (electrically erasable programmable ROM), and the like. The memory 13 stores system. programs and application programs executed by the CPU 11, data necessary for executing the programs, and the like.

The memory 13 also stores:

the motion data collected in running training and a race; and

the running index data derived from the motion data.

The display 14 constituted by LED lamps. The display 14 can display transmission status of data (for example, whether the data is being transmitted), on/off status of a GPS receiver, and the like.

The operation interface 15 includes:

a power button for turning on/off (not shown); and

a start/end button that orders start/end of data acquisition (not shown).

The CPU 11 controls the components based on commands from the operation interface 15.

The sensor 16 includes:

motion sensor that detects movement of the measurement device 10, such as three-axes acceleration sensor, a gyro sensor, and a geomagnetic sensor; and

a GPS receiver that acquires positional information of the measurement device 10.

The sensor 16 outputs measurement result to the CPU 11.

The communicator 17 transmits the running index data to the running analysis device 20 based on control by the CPU 11. The running index data is derived from motion data in running training and a race. The communicator 17 is, for example, a communicator that adopts a wireless standard such as Bluetooth (registered trademark), or a wired communicator such as a USE terminal.

### Running Analysts Device

Next, functional configuration of the running analysis device 20 will be described with reference to FIG. 4. FIG. 4 is a block diagram showing functional configuration of the running analysis device 20.

The running analysis device 20 includes a CPU 21, RAN 22, memory 23, a display 24, an operation interface 25, and a communicator 26. Components of the running analysis device 20 are connected via a bus 27.

The CPU 21 controls the components of the running analysis device 20. The CPU 11 reads a designated program among system programs and application programs stored in the memory 23 and develops them in the RAM 22. The CPU cooperates with the programs to execute processing of various kinds.

The RAM 22 is volatile memory and provides a work area where various data and programs are temporarily stored.

The memory 23 is constituted. by, for example, flash memory, EEPROM, an HDD (hard disk drive), or the like. The memory 23 stores system programs and application programs executed by the CPU 21, data necessary' for executing these programs, and the like.

The display 24 is constituted by an LCD (liquid crystal display), an EL (electro luminescence) display, or the like, and performs various displays according to display information ordered by the CPU 21.

The operation interface 25 includes:

various operation buttons (not shown) provided on a main body of the running, analysis device 20; and

a touch sensor (not shown) provided on the display 24.

The operation interface 25 receives user's input operation and outputs operation information to the CPU 21.

The communicator 26 receives the running index data from the measurement device 10. The communicator 26 is, for example, a communicator that adopts a wireless standard such as Bluetooth (registered trademark), or a wired communicator such. as a USE terminal.

### Operation of Measurement Device

Next, running index deriving processing and estimation processing of a waveform indicating a ground reaction. force, which. are operation of the measurement device 10, will be described. The running index derived by the running index deriving, processing has correlation with three indexes of a landing impact (hereinafter, referred to as force plate indexes). The three indexes are VIP (vertical impact peak), VALR (vertical average loading rate), and VILR (vertical instantaneous load rate), which are derived by means of a force plate. A waveform to be estimated in the estimation processing of a waveform indicating a ground reaction force is a waveform indicating a ground reaction force (i.e., a waveform indicating a reaction force in an up-down direction) which is used to derive the above three force plate indexes of a landing impact. Therefore, before explaining operation of the measurement device 10, the waveform indicating a ground reaction force and the three force plate indexes of a landing impact will be explained.

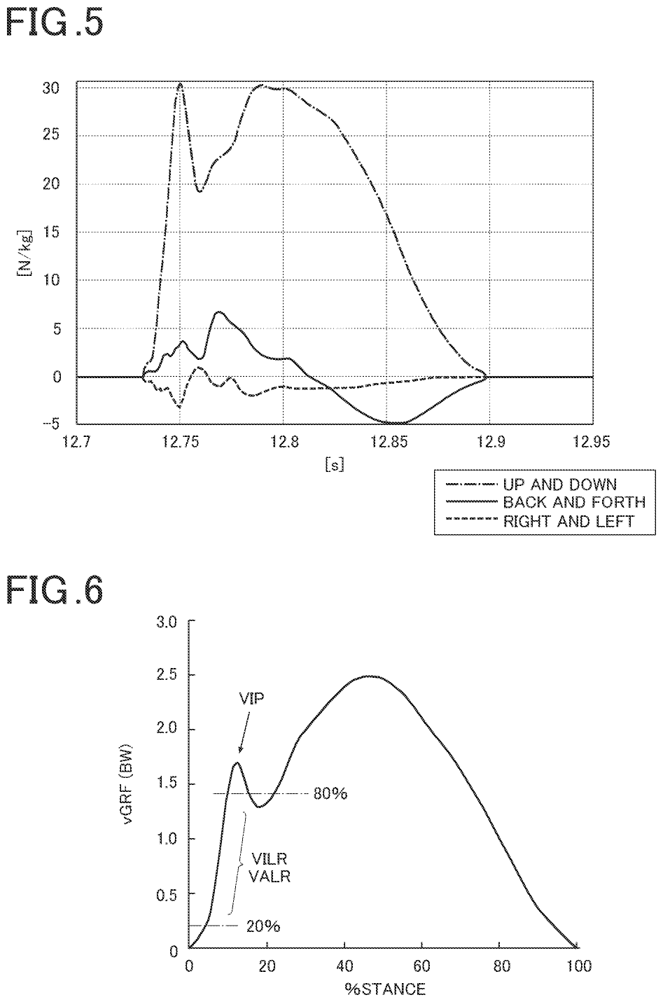

FIG. 5 is a graph showing measurement result of a reaction force when a foot of a running subject lands on the force plate. A waveform expressed with a broken line in the figure indicates a reaction force in a right-left direction of the running subject. A waveform expressed with a solid line indicates a reaction force in a back-forth direction of the subject. A waveform expressed with a dashed line indicates a reaction force in an up-down direction of the subject. As for the right-left direction, a leftward direction is positive while a rightward direction is negative. As for the back-forth direction, a backward direction is positive while a forward direction is negative. As for the up-down direction, an upward direction is positive while a downward. direction is negative. In the graph, values of measured reaction forces are normalized by being divided. by a weight. of the subject. That is, a unit “N/kg” of the vertical axis in the graph is equivalent to a unit “m/s²” that represents acceleration.

As shown in FIG. 5, a waveform showing a reaction force in the up-down direction (waveform expressed with a dashed line), that is, a waveform to be estimated in the estimation processing of a waveform indicating a ground reaction force (hereinafter referred to as a. force plate waveform), which will be described later, usually includes two peaks. The first one of these peaks (the peak on the left in the figure) is due to a force generated by a landing impact. The second peak (the peak on the right side of the figure) is due to a force generated when the subject propels the body. In a case where the landing impact is small, the force plate waveform may include only one peak.

FIG. 6 shows the three force plate indexes VIP, VALR, and VILR of a landing impact. The waveform shown in the figure is a normalized force plate waveform. The vertical axis of the graph. represents the ground reaction force in the vertical direction (up-down direction) with respect to a body weight (vGRF (BW); vertical ground reaction force (body weight)). On the horizontal axis, a landing time corresponds to 0% (STANCE) while an off-ground time of a foot to be landed corresponds to 100% (STANCE).

As shown in FIG. 6, VIP is a peak value of a first peak of the force plate waveform. VILR is a maximum value of an inclination in a period during which the VIP value rises from 20% to 80%. VALR is an average value of an inclination in a period during which the VIP value rises from 20% to 80%.

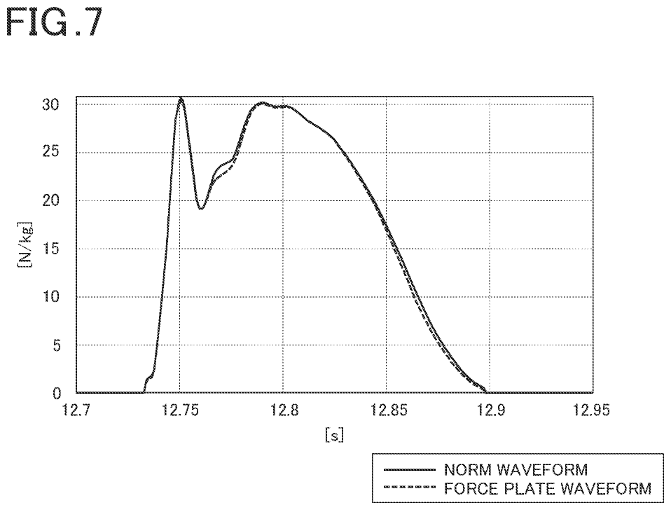

The graph of FIG. 7 shows the force plate waveform (i.e., the waveform showing the reaction force in the up-down direction) together with a norm waveform showing norms of reaction forces in three directions (i.e., the right-left direction, the back-forth direction, and the up-down direction). In the figure, the norm waveform is expressed with a solid line. The force plate waveform (i.e., the waveform showing the reaction force in the up-down direction) is expressed with a broken line.

As shown in FIG. 7, most of the norm waveform and the force plate waveform (i.e., the waveform showing the reaction force in the up-down direction) overlap. It is known from this that the reaction force in the up-down direction accounts for most of a floor reaction force (i.e., ground reaction force). To consider acceleration data (described later) in the up-down direction which is obtained by the measurement device 10, it makes it possible to reverse-estimate directional diffusion by means of a norm of the acceleration data.

### Running Index Deriving Processing

FIG. 3 is a flowchart showing control procedure of running index deriving processing. The running index deriving processing is started in response to, for example, user's pressing operation ordering start of data acquisition via the above-mentioned start/end button (the operation interface 15) at the beginning of running training.

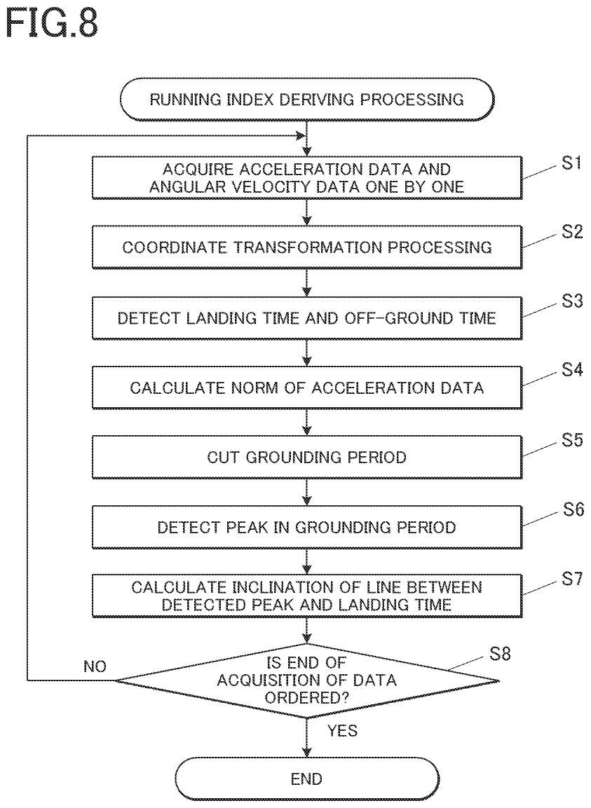

As shown, in FIG. 8, first, the CPU 11 of the measurement device 10 acquires (i) acceleration data detected by the three-axes acceleration sensor of the sensor 16 and (ii) angular velocity data detected by the gyro sensor, one by one (Step S1).

Next, the CPU 11 performs coordinate transformation processing (Step 32). The coordinate transformation processing transforms the acceleration data and the angular velocity data acquired in Step S1 into a world coordinate system from a sensor coordinate system. As shown in FIG. 2, among coordinates of the world coordinate system, the X-axis extends along the right-left direction of a running user. The Y-axis extends along the back-forth direction of the user. The Z axis extends along the up-down direction of the user. As for the X-axis, a leftward direction is positive while a rightward direction is negative. As for the Y-axis, a backward direction is positive while a forward direction is negative. As for the Z axis, an upward direction is positive while a downward direction is negative. Thus, the coordinate transformation processing into the world coordinate system from. the sensor coordinate system is performed on the acceleration data and the angular velocity data so that these data can be handled in the same coordinate system as that of the force plate indexes.

A method of transforming data into a world coordinate system from a sensor coordinate system is known. Explanation is omitted.

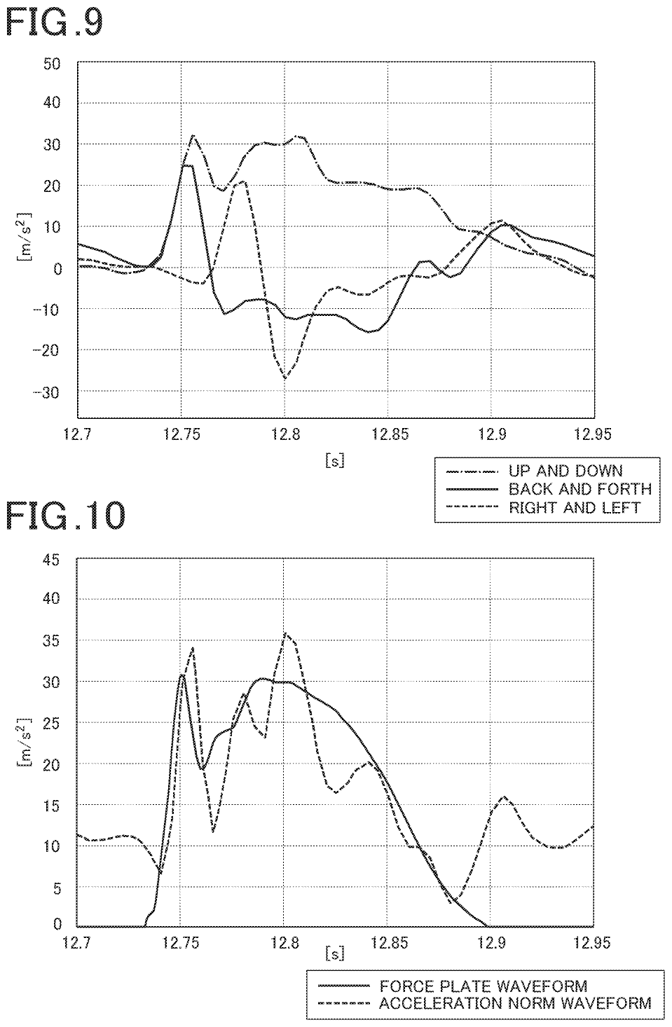

FIG. 9 shows a waveform or acceleration data on which coordinate transformation processing into a world coordinate system has been performed. The waveform expressed with a broken line in the figure shows acceleration data in the right-left direction (X-axis) of a running user. The waveform expressed with a solid line shows acceleration data in the back-forth direction (Y-axis) of the user. The waveform expressed with a dashed line shows acceleration data in the up-down direction (Z-axis) of the user.

As shown in FIG. 9, in the waveform. of the acceleration data in the up-down direction (Z-axis) of the user, it is difficult to recognize two peaks as compared with the above-mentioned force plate waveform. As can be known from this, the acceleration data is collected by the measurement device 10 mounted on a waist of the running user. While a force received from the ground. is transmitted a waist through. parts of the user's legs, shins, thighs, and hips, and parts of ankles, knees, and hip joints that connect those parts, a direction and time or the force are diffused.

Next, based on the acceleration data and the angular velocity data on which the coordinate transformation processing has been performed in Step S2, the CPU 11 detects (Step S3)

a landing time when a foot of the user touches the ground; and

an off-ground time when the foot of the user leaves the ground.

Methods of detecting the landing time and the off-ground time are disclosed in, for example, JP 2018-8015A. Explanation is omitted.

Next, the CPU 11 derives norms of pieces of the acceleration data on which. the coordinate transformation processing have been performed in Step S2, that is, pieces of acceleration data of the X-axis, Y-axis, and Z-axis (Step S4). Since the Z-axis acceleration data includes gravitational acceleration, the norm of the acceleration data is derived after subtracting a gravitational acceleration component from the acceleration data of the Z-axis.

The graph of FIG. 10 shows a waveform of the acceleration norm data derived in Step S4 together with the force plate waveform. In the figure, a solid line expresses the force plate waveform. A broken line expresses the acceleration norm data waveform.

As is known from FIG. 10, in a process in which a force received from the ground is transmitted to the measurement device 10 mounted on the user's waist, a landing impact component of the force received from the ground has a short time and is large. Therefore, the landing impact component is not so affected by body parts and joints between the user's feet and hips, and is transmitted as it is. On the other hand, a propulsion component of the force received from the ground is generated by moving joints with muscle of the user. Therefore, the propulsion component is affected by the joints and is much diffused. In view of this, the embodiment focuses on the landing impact component. There is not a large difference in the landing impact component between the force plate waveform and the waveform of the acceleration norm data. An inclination of the straight line between he first peak and a point indicating the landing time of the user used as an index (running Index) having correlation with the force plate

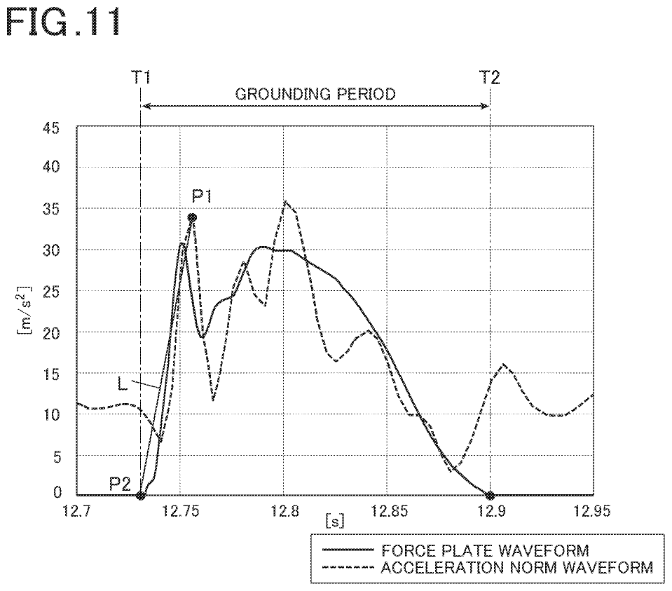

Next, the CPU 11 cuts out a grounding period (Step S5) between the landing time and the off-ground time from the waveform of the acceleration norm data which has been derived in Step S4. For example, as shown in FIG. 11, in a case where the time 12.73 s corresponds to the landing time T1 and the time 12.9 s corresponds to the off-ground time T2, a period between the landing time T1 and the off-ground time T2 is cut out as the grounding period.

Next, the CPU 11 detects the first peak of the waveform of the acceleration norm data (Step S6) in the grounding period cut out in Step S5. Specifically, as shown in FIG. 11, the CPU 11 sets a maximum point that first appears in the grounding period cut out in Step S5, that is, a first maximum point that appears after the user's landing time T1 in the grounding period, as the first peak P1.

A method of detecting the first peak is not limited to this. For example, maximum points (for example, four points) having larger maximum values may be picked from maximum points in the grounding period. The maximum point that appears first among them is detected as the first peak. Alternatively, maximum points (for example, four points) having more prominence may be picked from maximum points in the grounding period. The maximum point that appears first among them is detected as the first peak. From another point of view, an impact at the time when a user's foot lands (landing impact) works in a backward direction of the runner. Therefore, the largest maximum point in a period which begins from the landing time and in which an acceleration on the Y-axis is in the backward direction, that is, in a period during which a value of the acceleration data on the Y-axis is positive, may be detected as the first peak. Alternatively, among maximum points generated in a certain period from the landing time, for example, 70 ms, a maximum point having the largest maximum value may be detected as the first peak. The first peak may be detected after noise is removed by filtering the acceleration norm data. Further, the acceleration data may be filtered before the norm of the acceleration data is derived.

Next, the CPU 11 derives an inclination of a straight line as a running index (Step S7), the straight line being between:

the first peak detected. in Step S6; and

the landing point indicating the landing time in the grounding period in which the first peak is detected.

Specifically, as shown. in FIG. 11, the CPU 11 derives an inclination of the straight line L between the first peak P1 and the landing point P2 indicating the landing time Ti. As for the force plate waveform, the acceleration is zero at the landing time. As for the waveform of the acceleration norm data, the acceleration is not zero even before the landing time. Therefore, the acceleration at the landing time T1 is regarded as zero, and the inclination of the straight line L between the first peak P1 and the landing point P2 indicating the landing time T1 (acceleration 0 m/s²) is calculated. The inclination is used as an estimated value.

Next, the CPU 11 determines whether the user has performed pressing operation ordering end of data acquisition via the start/end button (the operation interface 15) (Step S8).

In a case where the CPU 11 determines in Step S8 that the user has not performed the pressing operation ordering end of data acquisition via the start/end button (the operation interface 15) (NO in Step S8), the CPU 11 returns processing to Step S1 and repeats processing from Step S1.

On the other hand, in a case where the CPU 11 determines in Step S8 that the user has performed the pressing operation ordering end of data acquisition via the start/end button (operation interface 15) (YES in Step SB), the CPU 11 ends the running index deriving processing. ESTIMATION PROCESSING OF WAVEFORM INDICATING GROUND REACTION FORCE

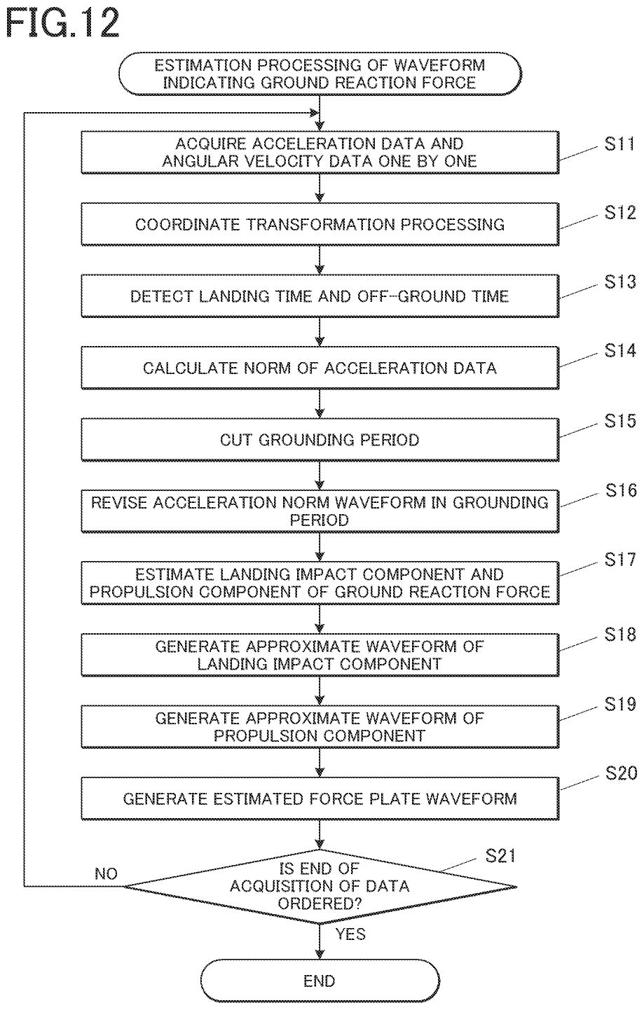

FIG. 12 is a flowchart showing control procedure of estimation processing of a waveform indicating a ground reaction force. Like the running index deriving processing, the estimation processing of a waveform indicating a ground reaction force begins in response to pressing operation ordering start of data acquisition by a user via the start/end button (the operation interface 15) at the beginning of running training or the like. Processing of steps S11 to S15 in the estimation processing of a waveform indicating a ground reaction force is the same as processing of steps S1 to S5 in the running index deriving processing. Explanation of processing of these steps is omitted, and processing from Step SI6 will be described.

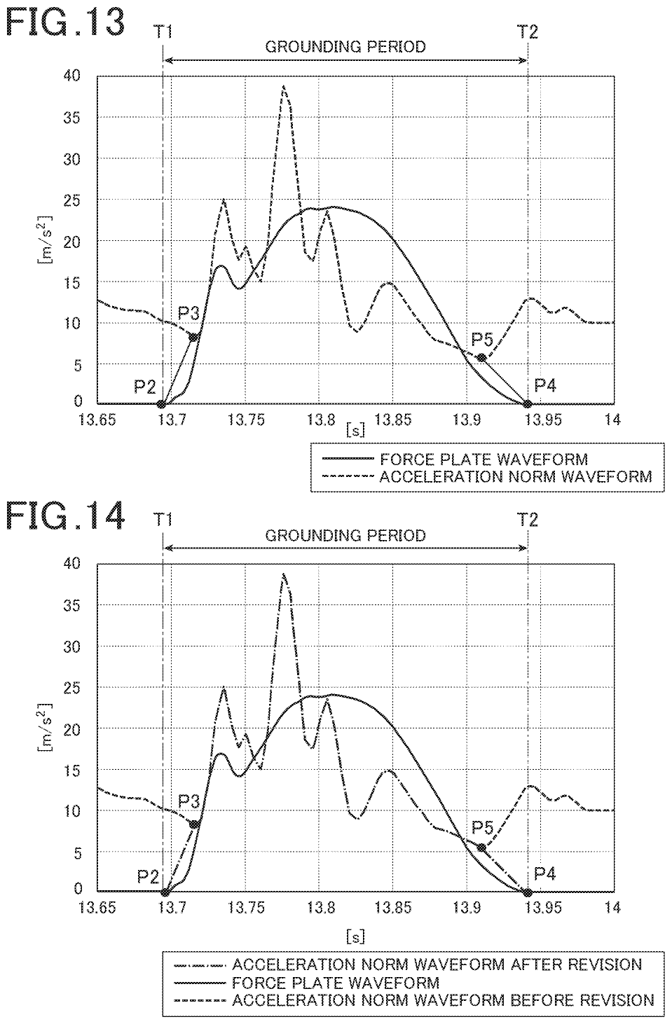

As shown in FIG. 12, in Step S16, the CPU 11 of the measurement device 10 revises the waveform of the acceleration norm data in the grounding period (Step S16). Specifically, as shown in FIG. 13, in a case where the grounding period between the landing time T1 at the time 13.69 s and the off-ground time T2 at the time 13.94 s is cut out, the CPU 11 regards a point where an acceleration at the landing time T1 is 0 as the landing point P2. The CPU 11 revises the waveform of the acceleration norm data with a line segment between the landing point P2 and a minimum point P3 that first. appears in the grounding period. Also, the CPU 11 regards a point where an acceleration at the off-ground time 12 is 0 as an off-ground point P4. The CPU 11 revises the waveform of the acceleration norm data by a line segment between the off-ground point P4 and a minimum point PS that appears last in the grounding period.

FIG. 14 shows the waveform of the acceleration norm data after revision. The waveform expressed with a dashed line the figure is the waveform. of the acceleration norm data after revision. The broken line toward the minimum. point P3 and the broken line from the minimum point P5 in the figure represent parts of the waveform of the acceleration norm data before revision. The waveform expressed with a solid line in the figure is the force plate waveform.

As shown by the waveform of the broken line in FIG. 14, the measurement device 10 is mounted on a waist of a user while being used, and acceleration is generated also outside the grounding period. Therefore, the waveform of the acceleration. norm. data is revised to be the waveform of a dashed line in the figure. The portion between the landing point P2 and the minimum point P3 and the portion between the off-ground point P4 and the minimum point P5 may be revised by spline interpolation.

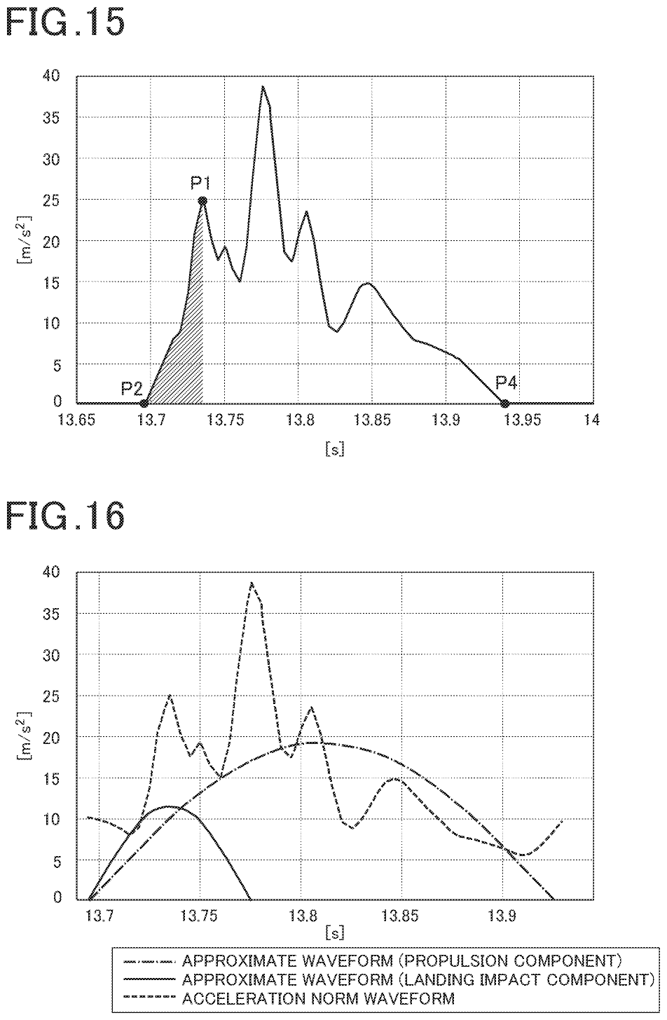

Next, the CPU 11 estimates the landing impact component and the propulsion component of a ground reaction force from the waveform of the acceleration norm data after revision (Step S17). Specifically, as shown in FIG. 15, the CPU 11 detects the first peak P1 in the waveform of the acceleration norm data after revision. The CPU 11 regards an integrated value from the landing point P2 to the first peak P1 as an amount of increase in the landing impact (shaded portion in the figure). The CPU 11 considers that there is the same amount of decrease as the amount of increase, and estimates the landing impact component as a value obtained by doubling the amount of increase. The CPU 11 subtracts the landing impact component from the integrated value from the landing point P2 to the off-ground point P4 in the revised waveform. The CPU 11 estimates the propulsion. component as the obtained value. The amount of increase in the landing impact, the landing impact component, and the propulsion component are represented by C1a, C1, and C2, respectively. The landing time (sampling point) , a first peak time (sampling point), and the off-ground time (sampling point) are represented by 0, t1, and t2, respectively. A value of the acceleration norm at each sampling point is represented by A(n). The amount C1a of increase in the landing impact, the landing impact component C1, and the propulsion component C2 are represented the Expressions (1), (2), and (3), respectively.

C1a=Σₙ₌₀^{t1}A(n) (1)

C1=2×C1a (2)

C2=Σₙ₌₀^{t2}A(n) (3)

Next, the CPU 11 generates an approximate waveform of the landing impact component (Step S18) estimated in Step S17.

Specifically, the CPU 11 first performs linear interpolation for the sampling points such that:

the sampling point of 0 (i.e., the landing time) corresponds to 0; and

the sampling point of t1 (i.e., the time of the first peak) corresponds to π/2; and

the sampling point of 2t1 corresponds to n.

Then, the CPU 11 substitutes values of sampling points (x is 0 to n) for which the linear interpolation is performed for “sinx”. The CPU 11 divides the sum of obtained values by the landing impact component Cl. Thereby the CPU 11 derives a coefficient k in an approximation expression. k·sinx to derive the approximation expression k·sinx. Then, as shown in FIG. 16, the CPU 11 generates an approximate waveform of the landing impact component (the waveform of a solid line in the figure) from the approximation expression k·sinx. The waveform expressed by a broken line in FIG. 16 is the waveform of the acceleration norm data.

Next, the CPU 11 generates an approximate waveform of the propulsion component estimated in Step S17 (Step S19).

Specifically, the CPU 11 first performs linear interpolation for the sampling points such that:

the sampling point of 0 (i.e., the landing time) corresponds to 0; and

the sampling point of t2 (i.e., the off-ground time) corresponds to π.

Then, the CPU 11 substitutes values of sampling points (x is 0 to π) for which the linear interpolation is performed for “sinx”. The CPU 11 divides the sum of obtained values by the propulsion component C2. Thereby the CPU 11 derives a coefficient “m” in an approximation expression m·sinx to derive the approximation expression m·sinx. Then, as shown in FIG. 16, the CPU 11 generates an approximate waveform of the propulsion component (the waveform of a dashed line in the figure) from the approximation expression m·sinx.

The CPU 11 then generates an estimated force plate waveform (Step S20).

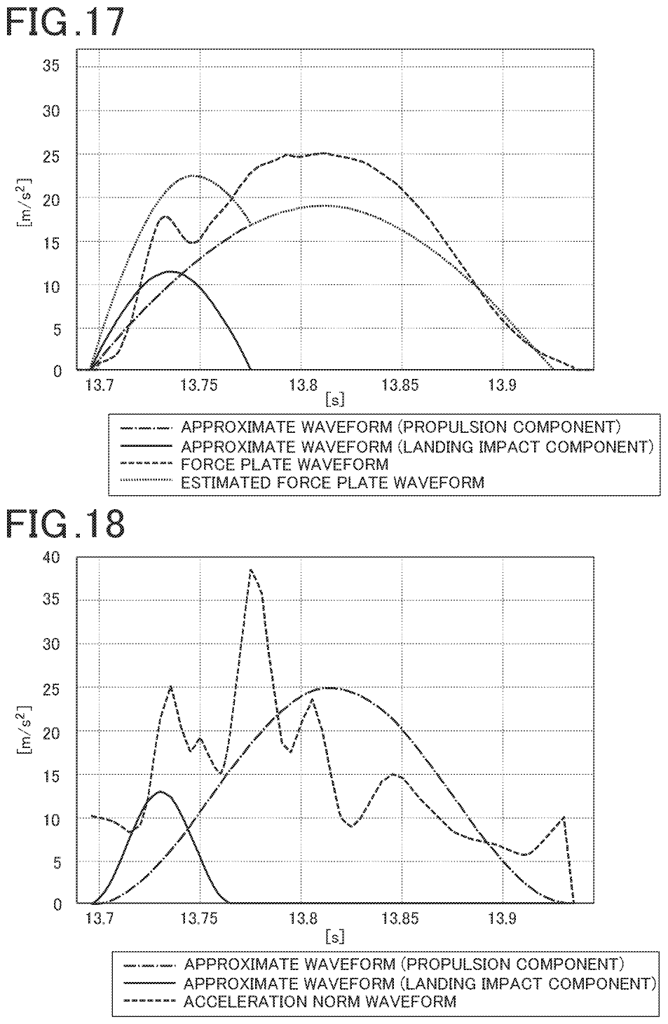

Specifically, as shown in FIG. 17, the CPU 11 generates the estimated force plate waveform. (the waveform of a dotted line in the figure) by synthesizing:

the approximate waveform of the landing impact component generated. in Step S18 (the waveform of the sold line in FIG. 16); and

the approximate waveform of the propulsion component generated in Step S19 (the waveform of the dashed line in FIG. 16).

The waveform expressed. by a broken line in FIG. 17 is the force plate waveform.

Next, the CPU 11 determines whether the user has performed pressing operation ordering end of data. acquisition via the start/end button (the operation interface 15) (Step S21).

In a case where the CPU 11 determines in Step S21 that the user has not performed the pressing operation ordering end of data acquisition via the start/end button (the operation interface 15) (NO in Step S21), the CPU 11 returns processing to Step S11 and repeats processing from Step S11.

In a case where the CPU 11 determines in Step S21 that the user has performed the pressing operation ordering end of data acquisition via the start/end button (the operation interface 15) (YES in. Step S21), the CPU 11 ends the estimation processing of a waveform indicating a ground reaction force.

In steps S18 to S19 of the estimation. processing of a waveform. indicating a ground reaction force, each of the landing impact component and the propulsion component is approximated with a sine wave. Alternatively, they may be approximated with cosine waves.

Specifically, as for the landing impact component, like approximation with a sine wave, the CPU 11 first performs linear interpolation for sampling points such that:

the sampling point of 0 (i the landing time) corresponds to 0;

the sampling point of t1 (i.e., the time of the first peak) corresponds to π/2; and

the sampling point of 2t1 corresponds to π.

Then, the CPU 11 substitutes values of the sampling points (x is 0 to π) for which the linear interpolation is performed for “1-cosx”. The CPU 11 divides the sum of obtained values by the landing impact component C1. Thereby the CPU 11 derives a coefficient k in an approximation expression (1-cosx) to derive the approximation expression (1-cosx). Then, as shown in FIG. 18, the CPU 11 generates an approximate waveform of the landing impact component (the waveform of a solid line in the figure) from the approximation expression k(1-cosx).

As for the propulsion component, like approximation with a sine wave, the CPU 11 first performs linear interpolation for sampling points such that:

the sampling point of 0 (i.e., the landing time) corresponds to 0; and

the sampling point of t2 (i.e., the off-ground time) corresponds to π.

Then, the CPU 11 substitutes values of the sampling points (x is 0 to π) for which the linear interpolation is performed for “1-cosx”. The CPU 11 divides the sum of obtained values by the propulsion component C2. Thereby the CPU 11 derives a coefficient “m” in an approximation expression m(1-cosx) to derive the approximation expression m(1-cosx). Then, as shown in FIG. 18, the CPU 11 generates an approximate waveform of the propulsion component (the waveform of a dashed line in the figure) from the approximation expression m(1-cosx).

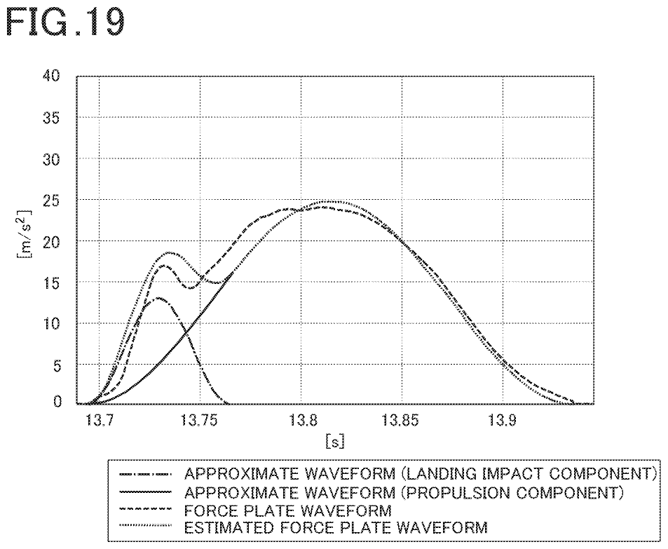

Then, as shown in FIG. 19, the CPU 11 generates an estimated force plate waveform (the waveform of a dotted line in the figure) by synthesizing:

the approximate waveform of the landing impact component (the waveform of the solid line in FIG. 18); and

the approximate waveform of the propulsion component (the waveform of the dashed line in FIG. 18).

For example, JP 2001-29329A filed in Japan discloses the floor reaction force measurement device in which the distribution measurement sheet is put on the upper side of the force. The distribution measurement sheet measures distribution of a reaction force on the force plate. However, according to the floor reaction force measurement device disclosed in JP 2001-29329A, place and space for installing the force plate are limited. Floor reaction forces of only several steps can be measured in one run. An index obtained from. data. of the floor reaction forces of the several steps (for example, an index related to the landing impact) is insufficient as information for analysis running.

On the other hand, the measurement device 10 of the embodiment acquires a peak (first peak) of secondary motion data for each predetermined period (grounding period) based on the secondary motion data. The secondary motion data indicates a level of primary motion data obtained while a user (target person) is running.

The measurement device 10 derives an index of the landing impact of the user for each predetermined period based on;

the acquired peak in the secondary motion data; and

the landing time of the user which is obtained from the primary motion data.

Therefore, according to the measurement device 10, the index of the landing impact the user is derived for each predetermined period. Sufficient indexes of the landing impact to be used in running analysis are obtained.

The measurement device 10 acquires a peak (first peak) in the waveform of the acceleration norm data for each predetermined period (grounding period).

Therefore, the measurement device 10 can obtain an index having correlation with the force plate indexes by deriving an index of the landing impact of the user based on:

the peak in the acceleration norm data; and

the landing time of the user which is obtained from the acceleration data.

The measurement device 10 acquires the first maximum point appearing after the landing time of the user as the peak (first peak) for each predetermined period (grounding period). Thereby the measurement device in can appropriately derive the index of the landing impact of the user.

The measurement device 10 derives, as an index related to the landing impact of a user (i.e., running index), an inclination of the straight line between:

the peak point indicating the acquired peak (first peak); and

the landing point that indicates the user's landing time.

Thus, the measurement device 10 can acquire a new index of a landing impact which has never existed before.

Based on the secondary motion data, the measurement device 10 acquires the peak (first peak) of the secondary motion data for each predetermined period (grounding period). The secondary motion data indicates a level of the primary motion data at a waist of a user.

Thus, the measurement device 10 can acquire the peak (first peak) in the secondary motion data by being installed on the waist of the user. The measurement device 10 can easily acquire the peak (first peak) in the secondary motion data. As a result, according to the measurement device 10, an index of a landing impact of a user can be derived one by one even in usual running, without using a conventional large-scale device. For example, changes in the index can be recognized in a race.

The description in the above embodiment is an example of the measurement device according to the present invention. The present invention is not limited. to this.

For example, in the estimation processing of a waveform indicating a ground reaction force according to the above embodiment, an approximate waveform. of the propulsion component is generated while a period of the propulsion component is regarded as a period between the landing time and the off-ground time, that is, the grounding period. Alternatively, for example, the approximate waveform may be generated while a period between the time of the first peak and the off-ground time is regarded as the period of the propulsion component.

In the estimation processing of a waveform indicating a ground reaction force according to the above embodiment, to generate an. approximate waveform of the propulsion component, the propulsion component is uniformly approximated with a sine wave or a cosine wave in the period of the propulsion component. Alternatively, for example, a peak of the propulsion. component may be obtained. An amount of increase from the landing time to the peak and an amount of decrease from the peak to the off-ground time may be approximated separately example, the amount of increase is approximated with a cosine wave while the amount of decrease is approximated with a sine wave.

In the running index deriving processing of the above embodiment, the inclination of the straight line between the point indicating the landing time of the user and the first peak is derived as the running index. In addition to this, the three force plate indexes of VIP, VALR, and VILR may be calculated by applying the inclination and a value of the first peak to a predetermined numerical expression.

The running index deriving processing and the estimation processing of a waveform indicating a ground reaction force in the above embodiment are executed by the CPU 11 of the measurement device 10. The present invention is not limited to this. For example, at least one CPU may execute a part of each processing. Specifically, for example, the measurement device 10 acquires the primary motion data and transmits the primary motion data to the running analysis device 20 via the communicator 17. Then, the CPU 21 in the running analysis device 20 performs the running index deriving processing and the estimation processing of a waveform indicating a around reaction force using the acquired primary' motion data.

Although embodiments of the present invention have been described and illustrated in detail, the disclosed embodiments are made for purposes of illustration and example only and not limitation. The scope of the present invention should be interpreted by terms of the appended claims.

What is claimed is:

1. An information processing device, comprising at least one processor that:
acquires a peak in secondary motion data for each of predetermined periods based on the secondary motion data indicating a level of primary motion data obtained from running or walking of a subject; and
derives an index of a landing impact of the subject for each of the predetermined periods based on:
the acquired peak in the secondary motion data; and
a landing time of the subject which is obtained from the primary motion data.

2. The information processing device according to claim 1, wherein
the primary motion data is acceleration data,
the secondary motion data is acceleration norm data which indicates a norm of the acceleration data, and
the at least one processor acquires a peak in a waveform of the acceleration norm data for each of the predetermined periods.

3. The information processing device according to claim 2, wherein the at least one processor detects a first maximum point after the landing time of the subject as a peak for each of the predetermined periods.

4. The information processing device according to claim 3, wherein the at least one processor derives, as the index, an inclination of a straight line between:
a peak point that indicates the acquired peak; and
a landing point that indicates the landing time of the subject.

5. The information processing device according to claim 4, wherein the at least one processor further derives a predetermined index of the landing impact based on the inclination, the predetermined index being derived with floor reaction force data obtained by a force plate.

6. The information processing device according to claim 1, wherein the secondary motion data indicates a level of the primary motion data at a waist of the subject.

7. The information processing device according to claim 1, wherein the at least one processor displays the derived index on the display of an external device connected to the information processing device.

8. An information processing method, comprising:
acquiring a peak in secondary motion data for each of predetermined periods based on the secondary motion data indicating a level of primary motion data obtained from running or walking of a subject; and
deriving an index of a landing impact of the subject for each of the predetermined periods based on:
the acquired peak in the secondary motion data; and
a landing time of the subject which is obtained from the primary motion data.

9. A recording medium storing a program that makes at least one processor of an information processing device:
acquire a peak in secondary motion. data for each of predetermined periods based on the secondary motion data indicating a level of primary motion data obtained from running or walking of a subject; and
derive an index related to a landing impact of the subject for each of the predetermined periods based on:
the peak in the secondary motion data which is acquired by the at least one processor; and
a landing time of the subject which is obtained from the primary motion data.
